(19) United States
(12) Patent Application Publication
(10) Pub. No.: US 2014/0243727 A1
(43) Pub. Date: Aug. 28, 2014

(54) HIGH-VISCOSITY SILICONE GEL ADHESIVE COMPOSITIONS

(71) Applicants: Dow Corning Corporation, Midland, MI (US); Dow Corning Taiwan Inc., Taipei (TW)

(72) Inventors: Roger A. Gibas, Bay City, MI (US); Yihan Liu, Midland, MI (US); Do-Lung Pan, Taoyuan Hsien (TW); Jeffrey T. Rastello, Saginaw, MI (US); Christine A. Weber, Pinconning, MI (US)

(73) Assignee: Dow Corning Corporation, Midland, MI (US)

(21) Appl. No.: 14/351,845

(22) PCT Filed: Oct. 12, 2012
(86) PCT No.: PCT/US2012/059996

§ 371 Date: Apr. 14, 2014

Related U.S. Application Data
(60) Provisional application No. 61/546,346, filed on Oct. 12, 2011.

(51) Int. Cl. A61L 15/58; B41F 15/00

(52) U.S. Cl. CPC A61L 15/585; B41F 15/00
USPC 602/55; 427/2.31; 101/129

(57) ABSTRACT

Provided in various embodiments are high viscosity, shear-thinning silicone compositions that can be pattern coated directly onto a substrate. The silicone compositions may be prepared by mixing at least one organopolysiloxane, at least one SiH-containing organopolysiloxane, at least one emulsifying agent, a hydrosilyation catalyst and water. The silicone compositions may be applied on a substrate for use in medical devices or wound dressings.

### FIELD OF THE INVENTION

The invention relates to high viscosity, shear-thinning silicone compositions that can be pattern coated directly onto a substrate. The high viscosity, shear-thinning silicone compositions exhibit adhesive properties and can be used, for example, in medical dressings and applications where a suitable skin-facing adhesive material is desired.

### BACKGROUND OF THE INVENTION

Most advanced wound care applications demand that exudate be removed from the patient's skin in order to prevent irritation and facilitate healing. While silicone gel adhesives are often used to provide some level of occlusiveness, locking in too much moisture over time can lead to wound maceration. The moisture level can be managed, to some degree, by making the silicone layer discontinuous. Several types of silicone dressings that have a discontinuous silicone layer have gained increasing acceptance in treating wounds such as pressure sores and ulcers. Conventional wound care products incorporate the use of polymeric foams, polymeric films, particulate and fibrous polymers, and/or non-woven and woven fabrics. Dressings with the right combination of these components promote wound healing by providing a moist environment, while removing excess exudate and toxic components, and further serve as a barrier to protect the wound from secondary bacterial infection.

However, these dressings often involve several layers of films and liners and complex preparation steps in order to produce a product that is capable of achieving the desired level of discontinuity while also retaining the desired level of adhesiveness in the silicone dressing. A typical silicone wound dressing construction starts with a multi-layer rollstock that contains a release liner, a silicone adhesive gel, an optional primer, a polyurethane film, and a paper liner. The paper liner is removed, and the silicone rollstock is then laminated on the absorbent media (such as a foam substrate), and topped with a suitable backing material. Additionally, many manufacturing processes employ further steps of perforating the carrier film to introduce holes into the film, further adding to the cost.

Therefore, what is needed in the art is a silicone coated wound dressing that can be prepared by a simpler, less expensive process that involves fewer materials while achieving the same or similar advantages of conventional silicone dressings. This invention answers that need.

### SUMMARY OF THE INVENTION

This invention relates to silicone compositions that are flowable in the presence of an applied stress and can be pattern coated directly onto a substrate. The silicone compositions exhibit high viscosity and shear-thinning properties.

The silicone composition may be prepared by mixing (a) at least one organopolysiloxane, (b) at least one SiH-containing organopolysiloxane, (c) at least one emulsifying agent, (d) a hydrosilyation catalyst and (e) water. A preservative may optionally be included in the silicone composition. The silicone composition is cured to form a silicone adhesive gel. The silicone composition exhibits (i) viscosity ranging from about 7000 cP to about 5,000,000 cP and (ii) shear thinning behavior, as determined by the rheological profile. Once the silicone composition is pattern coated onto a substrate, the pattern of the coating is able to be maintained upon application. It is contemplated that the water (component (e)) will be no greater than about 10 wt. % of the silicone composition to allow the silicone composition to maintain its pattern upon application. The silicone adhesive gel exhibits (i) adhesiveness ranging from about 0.2N to about 4N and (ii) cohesive strength, as determined by the peel adhesion test.

Additional aspects of the invention will be apparent to those of ordinary skill in the art in view of the detailed description of various embodiments, a brief description of which is provided below.

### DETAILED DESCRIPTION

This invention relates to a high viscosity, shear-thinning silicone composition that can be pattern coated directly onto a substrate prepared by mixing (a) at least one organopolysiloxane; (b) at least one SiH-containing organopolysiloxane; (c) at least one emulsifying agent; (d) a hydrosilyation catalyst; and (e) water. The high viscosity, shear-thinning silicone composition described herein has a relatively high resistance to flow. The high viscosity, shear-thinning silicone composition described herein is flowable in the presence of an applied stress and behaves more like a shear thinning gel.

The organopolysiloxane (component (a)) is an aliphatically unsaturated compound. The organopolysiloxane may have an average, per molecule, of one or more aliphatically unsaturated organic groups capable of undergoing hydrosilylation reaction. Alternatively, the organopolysiloxane may have an average of two or more aliphatically unsaturated organic groups per molecule.

The organopolysiloxane has the average formula (Formula I), R¹ₐSiO_{(4-a)/2}, where Formula I may be comprised of the following units: R¹₃SiO_{1/2} (building block M which represents a monofunctional unit); R¹₂SiO_{2/2} (building block D which represents a difunctional unit); R¹₁SiO_{3/2} (building block T which represents a trifunctional unit); or SiO_{4/2} (building block Q which represents a tetrafunctional unit). The number of building blocks (M, D, T, Q) in the organopolysiloxanes may range from 1 to 10,000, for instance from 4 to 1000.

Each of the open bonds from the oxygen atoms, designated as —O—, indicates a position where that building block may be bonded to another building block. Thus, it is through the oxygen atom that a first building block is bonded to a second or subsequent building block, the oxygen bonding either to another silicon atom or one of the R groups in the second or subsequent building block. When the oxygen atom is bonded to another silicon of the second building block, the oxygen atom represented in the first building block acts as the same oxygen atom represented in the second building block, thereby forming a Si—O—Si bond between the two building blocks.

At least one R¹ group is an aliphatically unsaturated group such as an alkenyl group. Suitable alkenyl groups contain from 2 carbon to about 6 carbon atoms and may be, but not limited to, vinyl, allyl, and hexenyl. The alkenyl groups in this component may be located at terminal, pendant (non-terminal), or both terminal and pendant positions. The remaining silicon-bonded organic groups in the alkenyl-substituted polydiorganosiloxane are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms and are may be, but not limited to, alkyl such as methyl, ethyl, propyl, and butyl; aryl such as phenyl; and halogenated alkyl such as 3,3,3-trifluoropropyl. In one embodiment, at least 50 percent of the organic groups in the alkenyl-substituted polydiorganosiloxane are methyl. The structure of the alkenyl-substituted polydiorganosiloxane is typically linear however; it may contain some branching due to the presence of trifunctional siloxane units.

Other suitable R¹ groups include, but are not limited to, acrylate functional groups such as acryloxyalkyl groups; methacrylate functional groups such as methacryloxyalkyl groups; cyanofunctional groups; monovalent hydrocarbon groups; and combinations thereof. The monovalent hydrocarbon groups may include alkyl groups such as methyl, ethyl, propyl, isopropyl, n-butyl, s-butyl, t-butyl, pentyl, neopentyl, octyl, undecyl, and octadecyl groups; cycloalkyl groups such as cyclohexyl groups; aryl groups such as phenyl, tolyl, xylyl, benzyl, and 2-phenylethyl groups; halogenated hydrocarbon groups such as 3,3,3-trifluoropropyl, 3-chloropropyl, dichiorophenyl, and 6,6,6,5,5,4,4,3,3-nonafluorohexyl groups; and combinations thereof. The cyano-functional groups may include cyanoalkyl groups such as cyanoethyl and cyanopropyl groups, and combinations thereof.

R¹ may also include alkyloxypoly(oxyalkyene) groups such as propyloxy(polyoxyethylene), propyloxypoly(oxypropylene) and propyloxy-poly(oxypropylene)-co-poly(oxyethylene) groups, halogen substituted alkyloxypoly(oxyalkyene) groups such as perfluoropropyloxy(polyoxyethylene), perfluoropropyloxypoly(oxypropylene) and perfluoropropyloxy-poly(oxypropylene) copoly(oxyethylene) groups, alkenyloxypoly(oxyalkyene) groups such as allyloxypoly(oxyethylene), allyloxypoly(oxypropylene) and allyloxy-poly(oxypropylene) copoly(oxyethylene) groups, alkoxy groups such as methoxy, ethoxy, n-propoxy, isopropoxy, n-butoxy and ethyihexyloxy groups, aminoalkyl groups such as 3-aminopropyl, 6-aminohexyl, 11-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups, hindered aminoalkyl groups such as tetramethyl piperidinyl oxypropyl groups, epoxyalkyl groups such as 3-glycidoxypropyl, 2-(3,4,-epoxycyclohexyl)ethyl, and 5,6-epoxyhexyl groups, ester functional groups such as acetoxymethyl and benzoyloxypropyl groups, hydroxyl functional groups such as hydroxy and 2-hydroxyethyl groups, isocyanate and masked isocyanate functional groups such as 3-isocyanatopropyl, tris-3-propyl isocyanu rate, propyl-t-butylcarbamate, and propylethylcarbamate groups, aldehyde functional groups such as undecanal and butyraldehyde groups, anhydride functional groups such as 3-propyl succinic anhydride and 3-propyl maleic anhydride groups, carboxylic acid functional groups such as 3-carboxypropyl, 2-carboxyethyl, and 10-carboxydecyl groups, metal salts of carboxylic acids such as zinc, sodium, and potassium salts of 3-carboxypropyl and 2-carboxyethyl groups, and combinations thereof.

Particular examples of organopolysiloxanes include polydimethysiloxane-polymethylvinylsiloxane copolymers, hexenyldimethylsiloxy-terminated polydimethylsiloxane-polymethylhexenylsiloxane copolymers, hexenyldimethylsiloxy-terminated polydimethylsiloxane polymers, vinyldimethylsiloxy-terminated polydimethylsiloxane polymers, vinyl or hexenyldimethylsiloxy-terminated poly(dimethylsiloxane-silicate) copolymers, mixed trimethylsiloxy-vinyldimethylsiloxy terminated poly(dimethylsiloxane-vinylmethylsiloxane-silicate) copolymers, vinyl or hexenyldimethylsiloxy terminated poly(dimethylsiloxane-hydrocarbyl) copolymers, derivatives thereof, and combinations thereof. Functional groups may be present at any point in the organopolysiloxane, for example, in the middle of the polymer or as an endgroup(s). Typical functional groups, such as diorgano-, —OH, -vinyl, -hexenyl, -epoxy, and -amine may be used in the organopolysiloxanes contemplated herein. End groups such as Me₃, Ph₂Me, Me₂Ph may or may not be present in the organopolysiloxane.

The SiH-containing organopolysiloxane (component (b)) is also known in the art as described, for example, in U.S. Pat. No. 3,983,298. The hydrogen atoms in this component may be located at terminal, pendant (non-terminal), or both terminal and pendant positions. The remaining silicon-bonded organic groups in this component are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms, and are exemplified by, but not limited to, alkyl such as methyl, ethyl, propyl, and butyl; aryl such as phenyl; and halogenated alkyl such as 3,3,3-trifluoropropyl. In one embodiment, at least 50 percent of the organic groups in the organosiloxane containing silicon-bonded hydrogen atoms are methyl. The structure of the organosiloxane containing silicon-bonded hydrogen atoms is typically linear however; it may contain some branching due to the presence of trifunctional siloxane units.

The SiH-containing organopolysiloxane has the average formula (Formula II), R²ₐSiO_{(4-a)/2}, where Formula II may be comprised of the following units: R²₃SiO_{1/2} (or building block M); R²₂SiO_{2/2} (or building block D); R²₁SiO_{3/2} (or building block T); or SiO_{4/2} (or building block Q). The number of building blocks (M, D, T, Q) in the organopolysiloxanes may range from 1 to 10,000, for instance from 4 to 1000. R¹ and R² are different because at least one R¹ has to be C═C and at least one R² has to be H.

Each of the open bonds from the oxygen atoms, designated as —O—, indicates a position where that building block may be bonded to another building block. Thus, it is through the oxygen atom that a first building block is bonded to a second or subsequent building block, the oxygen bonding either to another silicon atom or one of the R groups in the second or subsequent building block. When the oxygen atom is bonded to another silicon of the second building block, the oxygen atom represented in the first building block acts as the same oxygen atom represented in the second building block, thereby forming a Si—O—Si bond between the two building blocks.

In one embodiment, the number of building blocks (M, D, T, Q) in the SiH-containing organopolysiloxanes is from 1 to 1000. The SiH-containing organopolysiloxanes must contain at least one M, at least one D, or at least one T building block. In other words, the SiH-containing organopolysiloxanes cannot contain all Q building blocks. If there is only one building block, it can only be chosen from M, D, or T.

The SiH-containing organopolysiloxane may be a linear or cyclic compound containing from 1-10,000 (for instance, 1-1000, 1-200, or 1-100) of any combination of the following M, D, T, and Q building blocks. Examples of the SiH-containing materials described by Formula II that are useful in the methods described herein include oligomeric and polymeric organosiloxanes, such as (i) cyclic compounds containing 3-25 D building blocks (for instance, 3-10 or 4-6 D building blocks); or (ii) linear compounds containing two M building block that act an end blocks, and 2-10,000 D building blocks (for instance, 2-1000, 2-200, 10-100, 50-80, 60-70, 2-20, or 5-10) between the end blocks. Linear SiH-containing organopolysiloxanes may be particularly useful in some embodiments, for example, those containing combination(s) of pendant and terminal SiH groups.

The emulsifying agent (component (c)) may be any emulsifier known for emulsification of silicones and can be a cationic, anionic, nonionic, amphoteric and/or polymeric emulsifying agent/surfactant. Examples of suitable emulsifying agents or emulsifiers include synthetic surfactants, natural lipids and polymeric amphiphiles. Mixtures of emulsifiers of different types and/or different emulsifiers of the same type can be used. The emulsifier can be chosen to give optimum compatibility with the product into which the silicone emulsion is to be incorporated. Emulsifying agents having hydrocarbon lipophiles are generally suitable for silicone emulsions, depending on the solubility parameter of hydrocarbon lipophile.

Examples of suitable cationic emulsifiers include quaternary ammonium salts such as 8-22C alkyl trimethyl ammonium halides, particularly chlorides, 8-22C alkyl dimethyl benzyl ammonium halides or di(8-22C alkyl) dimethyl ammonium halides where the 8-22C alkyl group is for example octyl, decyl, dodecyl, hexadecyl, oleyl or octadecyl or tallow or coco alkyl groups, as well as corresponding salts of these materials, fatty amines and fatty acid amides and their derivatives, basic pyridinium compounds, quaternary ammonium bases of benzimidazolines and poly(ethoxylated/propoxylated) amines. Methosulphates, phosphates or acetates can be used as an alternative to halides.

Examples of suitable anionic emulsifiers include alkyl sulfates having at least 6 carbon atoms in the alky substituent such as sodium lauryl sulfate, sulfonic acids and their salts including alkyl, alkylaryl, alkylnapthalene, and aklyldiphenylether sulfonic acids and their salts having at least 6 carbon atoms in the alkyl substituent, such as dodecylbenzenesulfonic acid and its sodium or amine salt; long chain carboxylic acid surfactants and their salts such as lauric acid, steric acid, oleic acid and their alkali metal and amine salts, the sulfate esters of monoalkyl polyoxyethylene ethers, sulphonated glyceryl esters of fatty acids, salts of sulphonated monovalent alcohol esters, amides of amino sulphonic acids, sulphonated products of fatty acid nitriles, condensation products of naphthalene sulphonic acids with formaldehyde, alkali metal alkyl sulphates and ester sulphates, alkyl phosphates, sarcosinates and sulphonated olefins.

Examples of suitable nonionic emulsifiers include polyoxyalkylene alkyl ethers such as polyethylene glycol long chain (9-22C, especially 12-14C) alkyl ether, polyoxyalkylene sorbitan ethers, polyoxyalkylene alkoxylate esters, polyoxyalkylene alkylphenol ethers, ethylene oxide propylene oxide copolymers, polyvinyl alcohol, glyceride esters, alkyl glycosides and alkylpolysaccharides.

Examples of suitable amphoteric emulsifiers include cocamidopropyl betaine, cocamidopropyl hydroxysulphate, cocobetaine, sodium cocoamidoacetate, cocodimethyl betaine, N-coco-3-aminobutyric acid, imidazolinium carboxyl compounds and natural lipids.

The emulsifying agents can be added using suitable emulsification techniques available in the art. The emulsions that can be formed are oil-in-water emulsions. The emulsions that can be formed can also be non-aqueous emulsions; in other words, the water can be replaced by another polar solvent that is suitable for contacting skin and for use in medical dressings.

To form the silicone composition, the components (components (a), (b) (c) and (e)) are combined in the presence of a hydrosilyation catalyst (component (d)). Suitable hydrosilyation catalysts (d) include, but are not limited to, platinum catalysts such as chloroplatinic acid, alcohol solutions of chloroplatinic acid, dichlorobis(triphenylphosphine)platinum(II), platinum chloride, platinum oxide, complexes of platinum compounds with unsaturated organic compounds such as olefins, complexes of platinum compounds with organosiloxanes containing unsaturated hydrocarbon groups, such as Karstedts catalyst (i.e. a complex of chloroplatinic acid with 1,3-divinyl-1,1,3,3-tetramethyldisiloxane) and 1,3-diethenyl-1,1,3,3-tetramethyldisiloxane, and complexes of platinum compounds with organosiloxanes, wherein the complexes are embedded in organosiloxane resins. For example, a hydrosilyation catalyst may be a 0.5% platinum containing platinum-divinyltetramethyldisiloxane (a complex that is commercially available from Dow Corning Corporation in Midland, Mich.). The hydrosilyation catalyst may be added to the composition in an amount sufficient to provide, for example, 1 to 10 ppm of platinum based on the weight of the silicone composition.

Component (e) is water. In some embodiments, the water (e) is deionized water. The amount of water present is generally at least about 0.1 wt. % up to about 10 wt. % based on the total wt. % of the silicone composition. In further embodiments, the water may be present in amounts ranging from about 0.5 to about 10 wt. % based on the total wt. % of the silicone composition. It is contemplated that the water (component (e)) will be no greater than about 10 wt. % of the silicone composition to allow the silicone composition to maintain its pattern upon application.

If desired, other components can be added to the silicone composition including, but not limited to, fillers, pigments, low-temperature cure inhibitors, additives for improving adhesion, chain extenders, pharmaceutical agents, drugs, cosmetic agents, natural extracts, fluids or other materials conventionally used in gels, silicone fluids, silicone waxes, silicone polyethers, and rheology modifiers such as thickening agents.

One such optional component that can be included in the silicone composition is a preservative. Examples of suitable preservatives include formaldehyde, salicylic acid, phenoxyethanol, DMDM hydantoin (1,3-dimethylol-5,5-dimethyl hydantoin), 5-bromo-5-nitro-1,3-dioxane, methyl paraben, propyl paraben, sorbic acid, imidazolidinyl urea sold under the name GERMALL II (available from Sutton Laboratories in Chatham, N.J.), sodium benzoate, 5-chloro-2-methyl-4-isothiazolin-3-one sold under the name KATHON CG (available from Rohm & Haas Company in Philadelphia, Pa.), 1-(3-chloroallyl)-3,5,7-triaza-1-azoniaadamantane chloride sold under the trademark DOWACIL 75 (available from The Dow Chemical Company in Midland, Mich.), and iodopropynl butyl carbamate sold under the name GLYCACIL L (available from Lonza Incorporated in Fair Lawn, N.J.).

Where a preservative is included, the preservative may be present in any amount determined by one skilled in the art that would be sufficient to affect antimicrobial growth but not adversely impact the desired properties of the silicone adhesive gel described herein. Generally, the preservative may be present in amounts known to be effective by those skilled in the art. This range could, for example, range from about 0.01 to about 1.0 wt. % based on the total wt. % of the silicone composition. Where a preservative is used, the amount of preservative and type of preservative selected should be suitable for contacting skin and for use in medical dressings.

The organopolysiloxane (component (a)) and the SiH-containing organopolysiloxane (component (b)) may be present in any amount determined by one skilled in the art that would be sufficient to impart the desired properties of the silicone adhesive gel described herein. Generally, the SiH-containing organopolysiloxane to organopolysiloxane ratio ranges from about 0.8 to about 0.9.

The silicone composition exhibits (i) viscosity ranging from about 7000 cP to about 5,000,000 cP and (ii) shear thinning behavior, as determined by the rheological profile. Upon combining components (a), (b) (c), (d) and (e), the silicone composition is cured to form a silicone adhesive gel. The resulting silicone adhesive gel exhibits (i) adhesiveness ranging from about 0.2N to about 4N and (ii) cohesive strength, as determined by the peel adhesion test.

Viscosity of the silicone composition may be determined using a Brookfield viscometer or with a Helipath stand. The Brookfield viscometer measures viscosity by measuring the force required to rotate a spindle in fluid. The high viscosity silicone gel adhesive compositions contemplated herein have a viscosity ranging from about 7000 cP to about 5,000,000 cP. This viscosity range provides the silicone with viscosity that allows it hold a pattern when applied on a substrate without significantly absorbing into the substrate. Alternatively, the viscosity ranges from about 15,000 cP to about 5,000,000 cP, or from about 20,000 cP to about 5,000,000 cP. The application viscosity depends on the amount and type of shear applied.

In accordance with the Standard Test Method for Apparent Viscosity of Adhesives Having Shear-Rate-Dependent Flow Properties, ASTM-2556-93a (2005), the rheological properties of the silicone adhesive gel may be measured. Shear thinning or pseudoplastic behavior is the behavior exhibited when viscosity decreases with an increasing rate of shear stress. By analyzing the rheological profile of the silicone adhesive gel, it can be determined whether or not the silicone adhesive gel will exhibit shear thinning behavior.

Adhesion may be determined by peel adhesion tests. In accordance with the International Standard for Peel Adhesion of Pressure Sensitive Tape, PSTC-101 (issued October 2000 and last revised May 2007), peel adhesion tests show the pull-off adhesion strength of pressure sensitive tapes. For the purposes of this application, an adhesive gel that has low peel adhesion properties does not possess adhesiveness. When the adhesiveness drops much below 0.2N, it does not possess a sufficient amount of adhesiveness to act as an adhesive gel, for instance to adhere to the outside layer of a wound; when the adhesiveness increases much above 4N, the application and subsequent removal of the adhesive gel from the wound can become problematic or discomforting to the patient. Alternatively, the adhesiveness ranges from about 1.0N to about 3N; alternatively, from about 1.5 to about 3N.

Cohesive strength may be determined by peel adhesion tests. In accordance with the International Standard for Peel Adhesion of Pressure Sensitive Tape, PSTC-101 (issued October 2000 and last revised May 2007), peel adhesion tests show the pull-off adhesion strength of pressure sensitive tapes. For the purposes of this application, an adhesive gel that does not remain intact during the test does not possess cohesive strength.

It is contemplated that the silicone composition may be prepared as a multiple part (e.g., 2 part) composition, for example, when the composition will be stored for a long period of time before use. In the multiple part composition, the catalyst is stored in a separate part from any ingredient having a silicon bonded hydrogen atom, for example ingredient (b), and the various parts are combined shortly before use of the composition. For example, a two part composition may be prepared. In one such embodiment, the first part, Part A, may comprise at least one organopolysiloxane (ingredient (a)) in the presence of a hydrosilyation catalyst (ingredient (d)). Part A is emulsified with at least one emulsifying agent (ingredient (c)) and water (ingredient (e)) to create a stable emulsion having a desired particle size. The second part, Part B, may comprise at least one SiH-containing organopolysiloxane (ingredient (b)). Part B is emulsified with at least one emulsifying agent (ingredient (c)) and water (ingredient (e)) to create a stable emulsion having a desired particle size. A preservative may optionally be added to either the Part A emulsion or the Part B emulsion. The Part A emulsion may be combined with the Part B emulsion at ambient or elevated temperature to create a high viscosity, shear-thinning silicone composition. The Part A and Part B emulsions may be combined by any convenient means, such as mixing, shortly before use.

The silicone gel adhesive compositions described herein may be used as the skin-facing layer of a medical device or wound dressing. In addition to the silicone gel adhesive composition, the medical dressing contains an absorbable or porous substrate. The absorbable substrate may be any material known to those of skill in the art capable of at least partially absorbing the exudate from the wound. Absorbable substrates include, but are not limited to, the following materials: foams (e.g., polyurethane and/or polymer foams), synthetic sponges, natural sponges, silks, keratins (e.g., wool and/or camel hair), cellulosic fibers (e.g., wood pulp fibers, cotton fibers, hemp fibers, jute fibers, and/or flax fibers), rayon, acetates, acrylics, cellulose esters, modacrylics, polymers, super-absorbent polymers (e.g., polymers capable of absorbing approximately 10 times their weight or greater), polyamides, polyesters, polyolefins, polyvinyl alcohols, and/or other materials. Combinations of one or more of the above-listed materials may also be used as the absorbable or porous substrate.

The silicone gel adhesive compositions described herein may also be used as the skin-facing layer in various applications where suitable skin-facing adhesive materials are desired. Representative examples of additional skin-facing uses of the adhesive compositions described herein are in athletic apparel such as biking shorts and feminine hygiene products.

A medical dressing, as known to those of skill in the art, is an adjunct used by a person for application to a wound to promote healing and/or prevent further harm. A medical dressing is designed to be in direct contact with the wound, although, for the purposes of this application, direct contact on all areas of the wound is not necessary. Among other purposes, a medical dressing is designed to (a) stem bleeding and help to seal the wound to expedite the clotting process; (b) absorb exudate by soaking up blood, plasma and other fluids exuded from the wound; (c) ease pain of the wound; (d) debride the wound by removing the slough and foreign objects from the wound; (e) protect the wound from infection and mechanical damage; and (f) promote healing through granulation and epithelialization. A medical dressing comprising the silicone gel adhesive composition described herein, like other medical dressings, is designed to accomplish one or more of these design objectives.

It is also desirable for the medical dressing to retain a sufficient amount of moisture without retaining too much moisture, which can lead to an excessively wet environment for the wound which promotes the growth of bacteria, thus leading to wound maceration or other ailments. Balancing the moisture vapor is one way to gauge whether the dressing contains an appropriate amount of moisture. Other measures may also be used.

Making the silicone adhesive layer of the medical dressing discontinuous is one way to promote a balanced moisture vapor. Medical dressings can be made discontinuous in various ways, for instance by utilizing a perforated carrier material to create a path for exudate to pass through to the absorbent pad. One example of such a perforation process involves making small holes in the polyurethane carrier film to which the silicone gel adhesive composition is applied, then blowing air through the holes or using an ultrasonic device to open up the holes in the silicone layer while the composition cures.

Another means of making the silicone layer of a medical dressing discontinuous involves applying the silicone composition on the substrate in a pattern so that the pattern naturally creates discontinuity in the areas on the substrate that are not coated with the silicone composition. Similar to creating a carrier material with perforations, applying the discontinuous (or semi-continuous) pattern on the substrate creates a coating with void areas that allow exudate to pass through to the substrate to be absorbed. Any predetermined pattern that creates the void areas is sufficiently discontinuous for these purposes. The discontinuity of the pattern also enables an avenue for the moisture to be released from the wound, promoting a balanced moisture vapor. Accordingly, one contemplated embodiment relates to a silicone composition that has the ability to be pattern coated on a substrate such as an absorbable substrate; another embodiment relates to a medical dressing containing a substrate such as an absorbable substrate pattern coated with a silicone composition; and yet another embodiment to a method of preparing a medical dressing comprising the step of coating the silicone composition onto a substrate such as an absorbent substrate in a predetermined pattern.

The silicone composition may be applied to the substrate using any means known in the art, for instance through a screen printing or stenciling process. In the screen printing process, a screen or woven mesh is typically placed atop of the substrate, where the mesh contains a design that provides for an open area to transfer. The operator uses a roller or a squeegee to apply the silicone composition by pressing the gel through the mesh onto the substrate as the squeegee or roller is pushed to the rear of the screen. The thickness of the silicone composition is generally proportional to the thickness of the mesh or stencil. Thus, the thickness of the silicone composition that is applied or coated onto the substrate may be controlled by the screen or mesh that is used in the application process. A typical thickness of the silicone composition ranges from about 3 mil (76.2 μm) to about 20 mil (508 μm). In other instances, the typical thickness of the silicone composition may range from about 5 mil (127 μm) to about 15 mil (381 μm). In further instances, the typical thickness of the silicone composition may range from about 8 mil (203.2 μm) to about 12 mil (304.8 μm). Other thicknesses can also be used, depending on the desired result. As the squeegee moves toward the rear of the screen, the tension of the mesh pulls the mesh up away from the substrate, leaving the silicone composition on the substrate surface.

There are three common types of screen-printing presses: the “flat-bed,” “cylinder,” and “rotary,” with the rotary press being the most common. These processes can be used to apply the silicone composition described herein onto a substrate such as an absorbable substrate. Any screen-printing press may be used in these processes. In a typical rotary screen printing, a passing web is pressed by a press roller against a heated engraved roller, the cavities of which are filled by a liquid that is applied by a doctor blade. The applicator unit is a heated trough that is sealed off against the engraved roller by a spring steel doctor blade. Via pressure of the engraved roller against the substrate, the material is transferred onto the web and a patterned coating, which conforms with the configuration of the engraved roller, is achieved. Processes such as, but not limited to, reverse-offset and gravure-offset rotary screen printing techniques may be used to apply the silicone composition described herein onto the absorbable substrate.

Automated dispensers, such as those manufactured by Graco, Inc. in Minneapolis, Minn., may also be used to apply the silicone gel adhesive composition described herein onto the substrate. Automated dispensing units, such as those sold by Graco, Inc., offer a precise, positive displacement metering using double-acting cylinders and fluid inlet pressure to continuously reciprocate two connected cylinders. As the major volume cylinder (base) and minor volume cylinder (catalyst) reciprocate, they positively displace the two material components on ratio to the outlet ports. Static mixers are incorporated into the system to deliver a homogeneous mix of base and catalyst.

Once the silicone composition has been pattern coated, it is cured to produce the silicone adhesive gel and the water is removed. Curing takes place by heating at temperatures ranging from, for example, about 90° C. to about 150° C. for times ranging from, for example, about 2 minutes to about 6 minutes. Subsequent heating can be used to remove the residual water or the water can be removed simultaneously with curing. Additionally, curing and removal of the water can take place essentially at the same time that the silicone composition is applied to the substrate. Since the silicone composition has the ability to hold the pattern, the thickness of the silicone adhesive gel is essentially the same as the thickness of the pattern silicone composition.

One of the unique benefits of the silicone composition is its ability to be pattern coated directly onto the substrate in a manner where the pattern of the coating is maintained upon application. It is believed that the combination of properties exhibited by the silicone composition, including the adhesion, viscosity, cohesive strength, and rheology discussed above enable this feature. Advantageously, the silicone composition does not penetrate most absorbent substrates, or only penetrates the substrate minimally, while staying on the surface and maintaining the pattern. As discussed above, maintaining the pattern to create the voids provides the desired discontinuity, which in turn, allows the exudate to pass through to the substrate and promotes a balanced moisture vapor.

### EXAMPLES

### Release

For release testing, the release liner was secured in the bottom clamp and the adhesive coated non-woven fabric was placed in the top clamp. The clamps were pulled apart at 10 mm/s for 130 mm. The resultant force to pull the release liner from the adhesive coated non-woven fabric was averaged over 10 cm (excluding the first 2 cm and last 1 cm of the 13 cm pull) and measured in Newtons per centimeter (N/2.5 cm). The final release value is the average of 5 test strips.

### Adhesion

For adhesion testing, the release liner was removed from the coated non-woven fabric and the test strip was adhered to the frosted side of a 1.5 in×7 in (3.8 cm×17.8 cm) strip of polycarbonate (Lexan GE Product No. 8813-112D) using a 5 lb (2.3 kg) rubber coated roller making one stroke forward and one stroke back at a rate of 1 in/sec (2.5 cm/sec). The sample was allowed to sit at room temperature for 30 minutes. The polycarbonate was secured in the bottom clamp and the adhesive coated non-woven fabric was placed in the top clamp. The clamps were pulled apart at 10 mm/s for 130 mm. The resultant force to pull the polycarbonate from the adhesive coated polyester non-woven fabric was averaged over 10 cm (excluding the first 2 cm and last 1 cm of the 13 cm pull) and measured in Newtons per centimeter (N/2.5 cm). The final release value is the average of 5 test strips.

### Cohesion

Cohesion was evaluated during the adhesion testing by determining how much adhesive remained on the polycarbonate. Measurements of cohesive failure were made by estimating the percentage of adhesive remaining on the polycarbonate surface.

### Viscosity

The viscosity of Parts A and B was measured at room temperature on a Rheometric Scientific SR5000 stress rheometer. The viscosity was measured at a rate of 2 s⁻¹ for 60 seconds with the measurement taken at the 60 second mark (25 mm parallel plates 1.0 mm gap).

### Rheology

The rheology of Parts A and B was evaluated by performing a frequency sweep on a strain controlled rheometer, Rheometrics RDS-II, across a frequency range of 0.01 rad/s to 100 rad/s (log scale—2 points per decade) at 100% strain and 30° C. (25 mm parallel plates—gap=1.5 mm).

### Example 1

### Part A

52.99 grams of Dow Corning MG-7-9900 Soft Skin Adhesive Part A (an organopolysiloxane available from Dow Corning in Midland, Mich.), 0.28 grams of HOSTAPUR SAS 30 (a surfactant available from Clariant Corporation in Charlotte, N.C.), and 1.71 grams of deionized water were added to a Max300 sample cup of a FLACKTEK SPEEDMIXER, model DAC 600 FVZ (available from Flacktek Inc. in Landrum, S.C.). The contents were mixed in the SPEEDMIXER at 2500 rpms for 1 minute. This produced an emulsion with a median particle size of 7.8 microns as measured by a MALVERN MASTERSIZER 2000 Version 5.54 (available from Malvern Instruments, Ltd. in the United Kingdom) in the volume mode. Part A had a viscosity of 117,000 cP.

### Part B

52.99 grams of Dow Corning MG-7-9900 Soft Skin Adhesive Part B (an SiH-containing organopolysiloxane available from Dow Corning in Midland, Mich.), 0.28 grams of HOSTAPUR SAS 30, and 1.70 grams of deionized water were added to a Max300 sample cup of a FLACKTEK SPEEDMIXER, model DAC 600 FVZ. The contents were mixed in the SPEEDMIXER at 2500 rpms for 1 minute. This produced an emulsion with a median particle size of 8.3 microns, as measured by a MALVERN MASTERSIZER 2000 Version 5.54 in the volume mode. Part B had a viscosity of 113,000 cP.

Both parts were then combined in a 1:1 ratio and mixed in a FLACKTEK SPEEDMIXER for 48 seconds to combine them into a homogenous emulsion. The material was then pattern coated to 5-10 mil (0.13-0.25 mm) on polyester, non-woven fabric and foam and then cured at 90-130° C. for 4 minutes. The silicone composition was cured in place, keeping the open patterned design. Patterns were achieved using stencils and screens.

Samples for release and adhesion were prepared by mixing the two parts in a FLACKTEK SPEEDMIXER. The silicone composition was then coated to approximately 0.25 mm thickness on a non-woven fabric substrate using a table top coater and 0.38 mm shims. The coated substrate was cured in an oven for 4 minutes at 130° C. After removing the coated substrate from the oven, it was immediately covered with LDPE diamond embossed release liner using a 15 lb (6.8 kg) rubber coated roller. The sample was allowed a minimum of 16 hours to equilibrate prior to testing. The coated substrate was cut into 2.54 cm strip with a minimum of 12.7 cm in length.

Release and adhesion were evaluated using a Texture Analyzer with the Self Tightening Roller Grips attachment with the clamps set 25 mm apart. Release was 0.04 N/2.5 cm and adhesion was 1.64 N/2.5 cm. There was no cohesive failure.

The rheology results are summarized in Tables A and B below.

TABLE A
Rheology of Part A

| Freq | Eta* | G′ | G″ | | Strain | Time |
|---|---|---|---|---|---|---|
| (rad/s) | (P) | (dyn/cm²) | (dyn/cm²) | tan_delta | (%) | (s) |
| 0.01 | 31,923 | 59 | 314 | 5.30 | 99.86 | 331 |
| 0.032 | 14,316 | 92 | 443 | 4.83 | 99.86 | 1429 |
| 0.1 | 6,661 | 154 | 648 | 4.21 | 99.86 | 1774 |
| 0.316 | 3,735 | 402 | 1,111 | 2.76 | 99.86 | 1895 |
| 1 | 1,786 | 928 | 1,527 | 1.65 | 99.85 | 1932 |
| 3.162 | 714 | 1,416 | 1,758 | 1.24 | 99.85 | 1944 |
| 10 | 280 | 1,892 | 2,067 | 1.09 | 99.81 | 1950 |
| 31.623 | 115 | 2,517 | 2,609 | 1.04 | 99.26 | 1957 |
| 100 | 53 | 3,490 | 4,009 | 1.15 | 94.28 | 1961 |

TABLE B
Rheology of Part B

| Freq | Eta* | G′ | G″ | | Strain | Time |
|---|---|---|---|---|---|---|
| (rad/s) | (P) | (dyn/cm²) | (dyn/cm²) | tan_delta | (%) | (s) |
| 0.01 | 26,694 | 49 | 262 | 5.32 | 99.86 | 332 |
| 0.032 | 13,109 | 70 | 409 | 5.83 | 99.86 | 1430 |
| 0.1 | 6,030 | 142 | 586 | 4.12 | 99.86 | 1776 |
| 0.316 | 3,354 | 353 | 1,000 | 2.83 | 99.86 | 1899 |
| 1 | 1,631 | 827 | 1,406 | 1.70 | 99.85 | 1935 |
| 3.162 | 663 | 1,289 | 1,652 | 1.28 | 99.85 | 1947 |
| 10 | 263 | 1,742 | 1,967 | 1.13 | 99.81 | 1953 |
| 31.623 | 108 | 2,343 | 2,501 | 1.07 | 99.27 | 1960 |
| 100 | 51 | 3,274 | 3,877 | 1.18 | 94.32 | 1965 |

### Example 2

### Part A

93.83 grams of Dow Corning MG-7-9900 Soft Skin Adhesive Part A, 0.38 grams of DEHYTON PK 45 (a surfactant available from Cognis Corporation in Cincinnati, Ohio), and 5.78 grams of deionized water were added to a Max100 sample cup of a FLACKTEK SPEEDMIXER, model DAC 150 FVZ. The contents were mixed in the SPEEDMIXER at 3500 rpms for 30 seconds. This produced an emulsion with a mono-modal particle size distribution centered about a median particle size of 19.9 microns, as measured by a MALVERN MASTERSIZER 2000 Version 5.54 in the volume mode. Part A had a viscosity of 77,300 cP.

### Part B

93.85 grams of Dow Corning MG-7-9900 Soft Skin Adhesive Part B, 0.38 grams of DEHYTON PK 45, and 5.77 grams of deionized water were added to a Max100 sample cup of a FLACKTEK SPEEDMIXER, model DAC 150 FVZ. The contents were mixed in the SPEEDMIXER at 3500 rpms for 30 seconds. This produced an emulsion with a mono-modal particle size distribution centered about a median particle size of 21.9 microns, as measured by a MALVERN MASTERSIZER 2000 Version 5.54 in the volume mode. Part B had a viscosity of 73,100 cP.

Samples for release and adhesion were prepared by mixing the two parts in a FLACKTEK SPEEDMIXER. The silicone composition was then coated to approximately 0.25 mm thickness on a non-woven fabric substrate using a table top coater and 0.38 mm shims. The coated substrate was cured in an oven for 4 minutes at 130° C. After removing the coated substrate from the oven, it was immediately covered with LDPE diamond embossed release liner using a 15 lb (6.8 kg) rubber coated roller. The sample was allowed a minimum of 16 hours to equilibrate prior to testing. The coated substrate was cut into 2.54 cm strip with a minimum of 12.7 cm in length.

Release and adhesion were evaluated using a Texture Analyzer using the Self Tightening Roller Grips attachment with the clamps set 25 mm apart. Release was 0.03 N/2.5 cm after 1 day at room temperature and was 0.04 N/2.5 cm after 30 days at room temperature. Adhesion was 1.10 N/2.5 cm after 1 day at room temperature and was 1.15 N/2.5 cm after 30 days at room temperature. There was no cohesive failure.

The rheology results are summarized in Tables C and D below.

TABLE C
Rheology of Part A

| Freq | Eta* | G′ | G″ | | Strain | Time |
|---|---|---|---|---|---|---|
| (rad/s) | (P) | (dyn/cm²) | (dyn/cm²) | tan_delta | (%) | (s) |
| 0.01 | 12,021 | 21 | 118 | 5.55 | 99.86 | 331 |
| 0.032 | 6,424 | 29 | 201 | 6.97 | 99.87 | 1428 |
| 0.1 | 4,193 | 61 | 415 | 6.82 | 99.86 | 1775 |
| 0.316 | 2,338 | 203 | 711 | 3.49 | 99.86 | 1892 |
| 1 | 1,289 | 517 | 1,180 | 2.28 | 99.85 | 1930 |
| 3.162 | 569 | 1,004 | 1,494 | 1.49 | 99.85 | 1943 |
| 10 | 234 | 1,464 | 1,823 | 1.25 | 99.80 | 1952 |
| 31.623 | 99 | 2,045 | 2,362 | 1.16 | 99.23 | 1959 |
| 100 | 46 | 2,844 | 3,668 | 1.29 | 94.22 | 1964 |

TABLE D
Rheology of Part B

| Freq | Eta* | G′ | G″ | | Strain | Time |
|---|---|---|---|---|---|---|
| (rad/s) | (P) | (dyn/cm²) | (dyn/cm²) | tan_delta | (%) | (s) |
| 0.01 | 8,844 | 14 | 87 | 6.39 | 99.86 | 331 |
| 0.032 | 5,119 | 21 | 160 | 7.58 | 99.86 | 1428 |
| 0.1 | 3,347 | 43 | 332 | 7.68 | 99.86 | 1775 |
| 0.316 | 1,997 | 158 | 611 | 3.86 | 99.86 | 1895 |
| 1 | 1,098 | 389 | 1,027 | 2.64 | 99.85 | 1932 |
| 3.162 | 516 | 858 | 1,388 | 1.62 | 99.85 | 1946 |
| 10 | 217 | 1,321 | 1,718 | 1.30 | 99.80 | 1951 |
| 31.623 | 93 | 1,886 | 2,257 | 1.20 | 99.23 | 1959 |
| 100 | 44 | 2,620 | 3,590 | 1.37 | 94.21 | 1963 |

### Example 3

The viscosity of Comparative Sample C, Dow Corning MG-7-9900 Soft Skin Adhesive, was tested. The viscosity of Sample C was measured at room temperature on a Brookfield DV-II+Viscometer with a Helipath Stand (Model D). The viscosity was measured at RVT No. 5 at 50 rpm. The samples were vacuum de-aired prior to testing. Ten data points were acquired during the initial down cycle. The reported viscosity was an average of the ten data points. The viscosity levels ranged from 4,300-5,900 cP.

Unlike the formulations in Examples 1 and 2, Sample C readily soaked into both foam and fabric substrates.

While the invention is susceptible to various modifications and alternative forms, specific embodiments have been shown by way of example in the drawings and described in detail herein. It should be understood, however, that the invention is not intended to be limited to the particular forms disclosed. Rather, the invention is to cover all modifications, equivalents, and alternatives falling within the spirit and scope of the invention as defined by the appended claims.

1. A silicon pattern coating process for making a patterned silicone adhesive gel comprising:
(1) mixing
(a) at least one organopolysiloxane,
(b) at least one SiH-containing organopolysiloxane,
(c) at least one emulsifying agent,
(d) a hydrosilyation catalyst, and
(e) water
to form a silicone composition, wherein the water comprises no greater than about 10 wt. % of the silicone composition
and the silicone composition exhibits
i. viscosity ranging from about 7000 cP to about 5,000,000 cP, and
ii. shear thinning behavior, as determined by the rheological profile;
(2) pattern coating the silicone composition onto an absorbent substrate in a predetermined pattern;
(3) curing the silicone composition to form a patterned silicone adhesive gel which maintains the predetermined pattern, wherein the patterned silicone adhesive gel exhibits;
i. adhesiveness ranging from about 0.2N to about 4N, and
ii. cohesive strength, as determined by the peel adhesion test.

2. The pattern coating process of claim 1, further comprising mixing (f) a preservative with the at least one organopolysiloxane, the at least one SiH-containing organopolysiloxane, the at least one emulsifying agent, the hydrosilyation catalyst and the water.

3. The pattern coating process of claim 1, wherein the at least one emulsifying agent is a cationic emulsifier, an anionic emulsifier, a nonionic emulsifier, or an amphoteric emulsifier.

4. The pattern coating process of claim 1, wherein the at least one emulsifying agent forms an oil-in-water emulsion.

5-9. (canceled)

10. A method of preparing a medical dressing containing a patterned silicone adhesive gel has been pattern coated thereon, comprising
(1) mixing (a) at least one organopolysiloxane, (b) at least one SiH-containing organopolysiloxane, (c) at least one emulsifying agent, (d) a hydrosilyation catalyst and (e) water to form a silicone composition, wherein the water comprises no greater than about 10 wt. % of the silicone composition and wherein the silicone composition exhibits
i. viscosity ranging from about 7000 cP to about 5,000,000 cP, and
ii. shear thinning behavior, as determined by the rheological profile;
(2) pattern coating the silicone composition onto an absorbent substrate of the medical dressing in a predetermined pattern; and
(3) curing the silicone composition to form a patterned silicone adhesive gel, wherein the patterned silicone adhesive gel exhibits:
i. adhesiveness ranging from about 0.2N to about 4N, and
ii. cohesive strength, as determined by the peel adhesion test
and maintains the predetermined pattern upon application.

11. The method of claim 10, further comprising mixing (f) a preservative with the at least one organopolysiloxane, the at least one SiH-containing organopolysiloxane, the at least one emulsifying agent, the hydrosilyation catalyst and the water.

12. The method of claim 10, wherein the predetermined pattern is discontinuous.

13. The method of claim 10, wherein the pattern coating is accomplished via a screen printing process or a stenciling process.

14. A method of preparing a medical device comprising a skin-facing layer that contains a patterned adhesive gel, comprising:
(1) mixing (a) at least one organopolysiloxane, (b) at least one SiH-containing organopolysiloxane, (c) at least one emulsifying agent, (d) a hydrosilyation catalyst and (e) water to form a silicone composition, wherein the water comprises no greater than about 10 wt. % of the silicone composition and wherein the silicone composition exhibits
i. viscosity ranging from about 7000 cP to about 5,000,000 cP, and
ii. shear thinning behavior, as determined by the rheological profile;
(2) pattern coating the silicone composition onto an absorbent substrate of the skin-facing layer of the medical device in a predetermined pattern; and
(3) curing the silicone composition to form a patterned silicone adhesive gel, wherein the patterned silicone adhesive gel exhibits:
i. adhesiveness ranging from about 0.2N to about 4N, and
ii. cohesive strength, as determined by the peel adhesion test thereby allowing and maintains the predetermined pattern to be maintained upon application.

15. The method of claim 14, further mixing (f) a preservative with the at least one organopolysiloxane, the at least one SiH-containing organopolysiloxane, the at least one emulsifying agent, the hydrosilyation catalyst and the water.

16. The method of claim 14, wherein the predetermined pattern is discontinuous.

17. The method of claim 14, wherein the pattern coating is accomplished via a screen printing process or a stenciling process.

18. The pattern coating process of claim 1, wherein the predetermined pattern is discontinuous.

19. The pattern coating process of claim 1, wherein the pattern coating is accomplished via a screen printing process or a stenciling process.

20. The pattern coating process of claim 19, wherein the screen printing process is a flat-bed process, a cylinder process or a rotary process.

21. The method of claim 10, wherein the at least one emulsifying agent is a cationic emulsifier, an anionic emulsifier, a nonionic emulsifier, or an amphoteric emulsifier.

22. The method of claim 10, wherein the at least one emulsifying agent forms an oil-in-water emulsion.

23. A medical dressing comprising a patterned silicone adhesive gel prepared by the method of claim 10.

24. The method of claim 14, wherein the at least one emulsifying agent forms an oil-in-water emulsion.

25. A medical device comprising a skin-facing layer that contains a patterned silicone adhesive gel prepared using the method of claim 14.
